(19) United States
(12) Patent Application Publication
(10) Pub. No.: US 2006/0200237 A1
(43) Pub. Date: Sep. 7, 2006

(54) Vertebral motion preservation device with improved rotational motion

(76) Inventor: Farid Bruce Khalili, Briarcliff Manor, NY (US)

Correspondence Address: LAWRENCE CRUZ, 12 GOOD HILL RD., BETHEL, CT, 06801, US

(21) Appl. No.: 11/062,512

(22) Filed: Feb. 22, 2005

(51) Int. Cl. A61F 2/44

(52) U.S. Cl. 623/17.11

(57) ABSTRACT

An implantable vertebral motion preservation system has upper and lower parts that are inserted into a vertebral space, wherein the upper part or lower part has a generally arcuate raised ridge that forms an arcuate shape and the other of the upper or lower part has a corresponding generally arcuate groove that receives the ridge. The ridge and groove are positioned with respect to the upper and lower parts, as well as the adjacent vertebrae, to share a common imaginary central axis such that during twisting motion of the body the upper and lower parts rotate relative to each other about said axis which sliding relative to each other along the ridge and groove contact areas. The cross-sectional geometry of the groove portion is curved so that during lateral bending movement of the vertebral column the ridge portion and attached upper or lower part follows a path of rotation and arcuate translation that corresponds to the cross-sectional shape of the groove portion, resulting in ease of movement and recoverable motion that improves return to the original position. During rotational movement of the vertebral column, as may result from typical twisting movement of the body, the adjacent upper and lower parts, as well as the adjacent vertebrae engaged to each, undergo relative rotational movement about the imaginary axis to result in ease of movement and recoverable motion that improves return to the original position.

### RELATED APPLICATIONS

None.

### TECHNICAL FIELD

The present invention relates to surgically implanted devices and, more particularly, implantable devices for vertebral repair or reconstruction with motion preservation aspects.

### BACKGROUND OF THE INVENTION

Various known techniques and systems exist for repairing or reconstructing injured or diseased vertebral sections in which one or more implants are provided in or adjacent to a vertebral disc space in a manner in which two adjacent vertebrae are fused together. Such a solution provides stability and pain relief, but has the potentially adverse effect of disabling movement between the fused vertebrae. In addition to limiting a patient's overall mobility, resultant stress increases occur at the opposite, or non-fused, ends of the adjacent vertebrae that are fused to each other. Thus, in certain circumstances, a system which preserves motion, as opposed to fusion, is desired.

Various known systems exist for joining adjacent vertebrae in a manner which preserves motion. Referring to FIG. 1A and FIG. 1B, for example, schematically shown are front end and partial top views, respectively, of a vertebral implant system (10). The system (10) comprises an upper part (12) and a lower part (14), each being adapted to be inserted into a disc space between adjacent vertebrae so that the upper part engagement portions (16) engage the lower part engagement portions (18). FIG. 1A depicts a situation in which such a system undergoes lateral bending of the vertebral column during natural movement of the body of a patient having such a system implanted. FIG. 1B depicts a situation in which such a system undergoes rotational movement of the vertebral column during natural movement of the body of a patient having such a system implanted. As can be seen in these schematics, the amount of relative movement between the upper part (12) and the lower part (14), in either instance, can be to a degree in which the corresponding engagement portions (16, 18) move out of contact and out of alignment with each other to a degree that makes return to the original position difficult and, sometimes, that prevents return to the original position.

### OBJECTS AND SUMMARY OF THE PRESENT INVENTION

It is desirable, therefore, to provide a system and technique of vertebral implant for securing adjacent vertebrae in a manner that preserves motion while overcoming the shortcomings of known systems, as described above, as well as other shortcomings. This object and other objects and advantages are inherent to the present invention described herein.

The present invention is directed to an implantable system having upper and lower parts that are inserted into a vertebral space such that the upper and lower parts engage each other and connect two adjacent vertebrae. The upper and lower parts may each be made of unitary bodies or one or both may be made from modular components that are constructed together as they are inserted into a patient to accommodate minimally invasive surgical techniques and to facilitate posterior approach insertion. The upper part or lower part has a generally arcuate raised ridge that forms a “u” shape or partial “u” shape. The other of the upper or lower part has a corresponding generally arcuate groove portion that receives the ridge. Alternatively, instead of a single raised ridge, a plurality of raised ridge-like or semi-spherical bumps may be used in an arcuate array. The ridge and groove are positioned with respect to the upper and lower parts, as well as the adjacent vertebrae, to share a common imaginary central axis such that during twisting motion of the body the upper and lower parts rotate relative to each other about said axis while sliding relative to each other along the ridge and groove contact areas. Additionally, the cross-sectional geometry of the groove portion is curved. The outer perimeter wall may be of greater height than the inner perimeter wall. During lateral bending movement of the vertebral column, as may result from typical bending movement of the body, the ridge portion and attached upper or lower part follows a path of rotation and arcuate translation that corresponds to the cross-sectional shape of the groove portion, resulting in ease of movement and recoverable motion that improves return to the original position. During rotational movement of the vertebral column, as may result from typical twisting movement of the body, the adjacent upper and lower parts, as well as the adjacent vertebrae engaged to each, undergo relative rotational movement about the imaginary axis in a smooth manner that is aided by the cooperation of the ridge and groove to result in ease of movement and recoverable motion that improves return to the original position.

### BRIEF DESCRIPTION OF THE DRAWINGS

FIG. 1A is a schematic, front end view of a prior art vertebral motion preservation system.

FIG. 1B is a schematic, partial top view of the system shown in FIG. 1A.

FIG. 2A is a schematic, front end view of a vertebral motion preservation system according to the present invention.

FIG. 2B is a schematic, partial top view of the system shown in FIG. 2B.

FIG. 3A is a schematic, top view of a first embodiment of the present invention shown inserted over a vertebral body.

FIG. 3B is a schematic, cross-sectional front view of the first embodiment of the present invention.

FIG. 3C is a schematic, partial cross-sectional view of the first embodiment of the present invention.

FIG. 4 is a perspective, disassembled, schematic view of the first embodiment of the present invention.

FIG. 5A is a top, schematic view of a second embodiment of the present invention.

FIG. 5B is a cross-sectional, partial, schematic view of the second embodiment of the present invention.

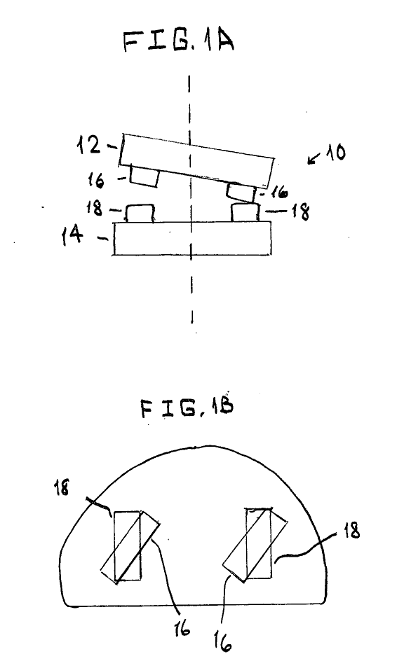

### DESCRIPTION OF THE PREFERRED EMBODIMENTS

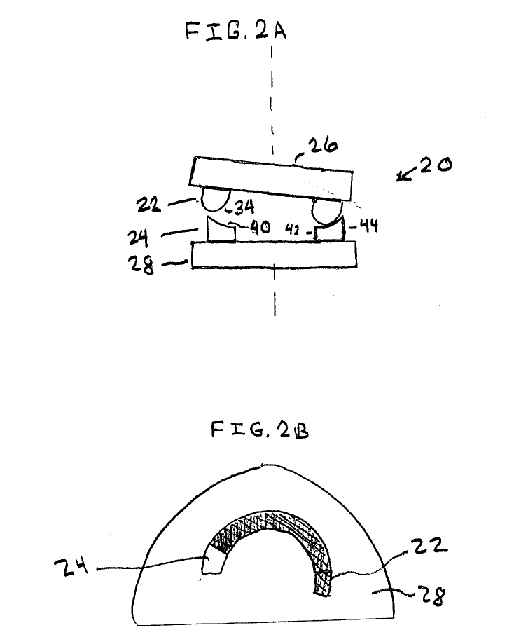

A first embodiment of the present invention is shown in FIGS. 2A-4. An intervebral implant system (20) comprises an upper part (22) and a lower part (24). The top surface of the upper part (22) may be provided with one or more various contours (not shown) of a type generally known to those skilled in the art to facilitate engagement and securing to a lower end face of a first vertebra (26). The bottom surface of the lower part (24) may be provided with one or more various contours (not shown) of a type generally known to those skilled in the art to facilitate engagement and securing to a lower end face of a second vertebra (28), which is located adjacent to and below the first vertebra (26). Each of the upper part (22) and lower part (24) may be made of suitable, known materials such as titanium, steel, alloys, ceramics, polymers or glass-like materials. The upper part (22) has, on its lower surface a rounded ridge (34) having, in cross-section, a semi-circular or dome profile. The lower part (24) has, on its upper surface a groove (40) which, in cross-section, is concave and has a wall height that is smaller at the inner side (42) of the “u” shape and a wall height that is greater the outer side (44) of the “u” shape.

When the upper and lower parts (22, 24) are positioned one on top of the other, as in a surgical construct between adjacent vertebrae, the ridge (34) fits into the groove (40) as shown in FIG. 2A. In FIG. 2A, the upper part ridge (34) is shown in cross-section in order to allow illustration of the ridge (34) positioned in the groove (40). In FIG. 2A, the ridge (34) is shown in a position rotated relative to the groove (40), as when relative rotation between the upper and lower parts (22, 24) occurs, as is discussed below.

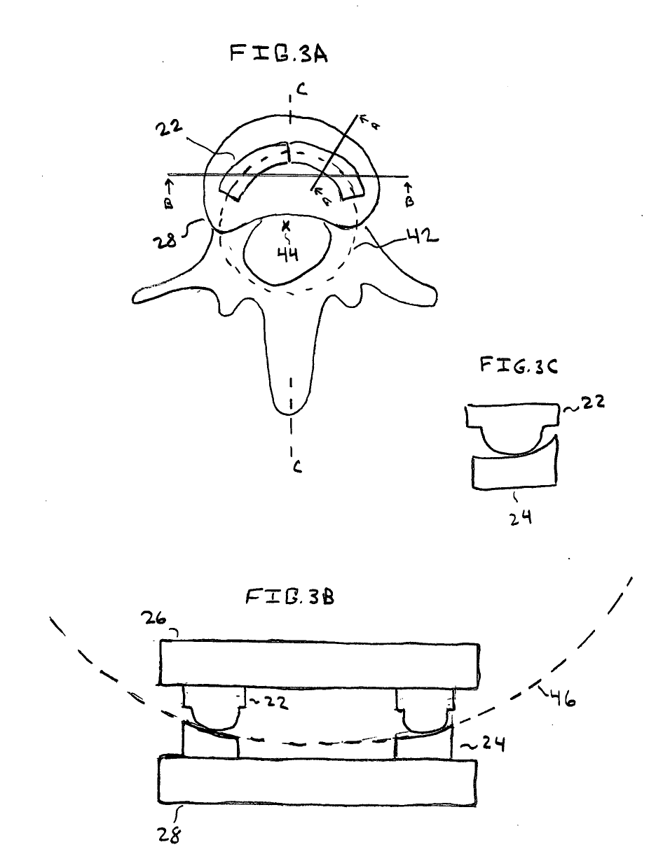

FIG. 3A illustrates the upper and lower parts (22, 24) assembled and positioned between adjacent vertebrae (26, 28), whereby in FIG. 3A the upper vertebra (26) is made transparent to allow illustration of the position of the upper and lower parts (22, 24). FIG. 3B is a cross-sectional view of the embodiment shown in FIG. 3A, taken along B-B. FIG. 3C is a partial, cross-sectional view of the same embodiment taken along A-A. As can be seen in FIG. 3A, the arcuate shape of the upper and lower parts (22, 24) form an imaginary circle (42) represented by a dashed line. The center (44) of the imaginary circle (42) represents an axis of rotation if the ridge (34) and groove (40) engage and slide relative to each other. In the end view of FIG. 3B, it can be seen that the curved cross-sectional shape of the groove (40) forms an imaginary arcuate path (46) along which relative movement between the ridge (34) and groove (40) can occur. These two imaginary paths (42, 46) represent relative rotational movement between upper and lower parts (22, 24) in two planes oriented generally orthogonally to each other. Relative rotation in a plane represented by C-C in FIG. 3A occurs by pivoting between the two parts (22, 24) at one end with temporary spacing or disengagement occurring at the other end.

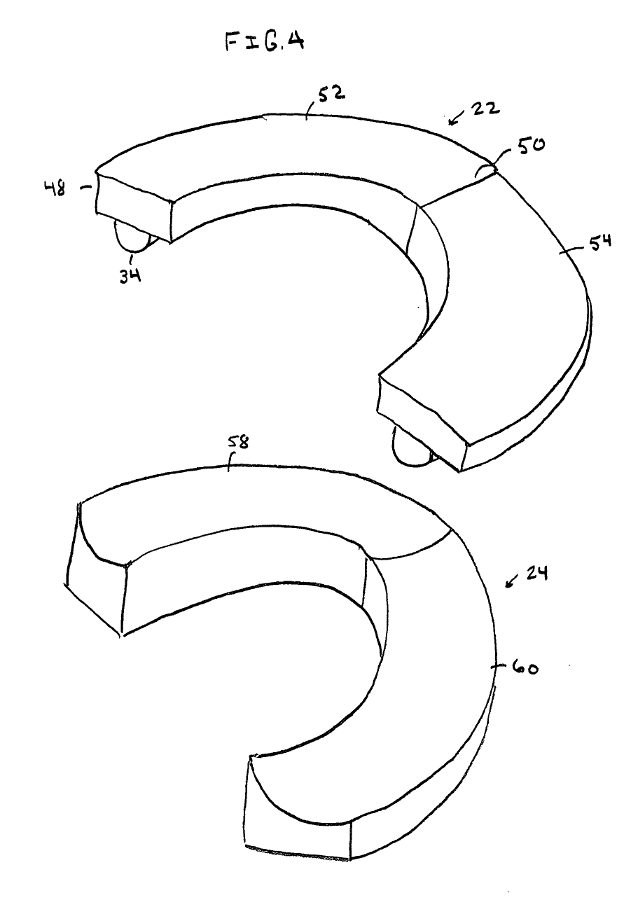

FIG. 4 is a perspective view of the upper part (22) and lower part (24). The raised ridge (34) may be integral with the remaining body portion (48), or it may be modular and selectively attachable and detachable. A line (50) shows where the upper part (22) may be separated into two halves, as is preferable, to enable a surgeon to install each half (52, 54) in separate steps, thus enabling a less invasive procedure than if the upper part (22) were unitary. The two halves (52, 54) are preferably joined by interlocking or interfitting contours or tabs on each end face (not shown). Similarly, the lower part (24) is preferably formed into two halves.

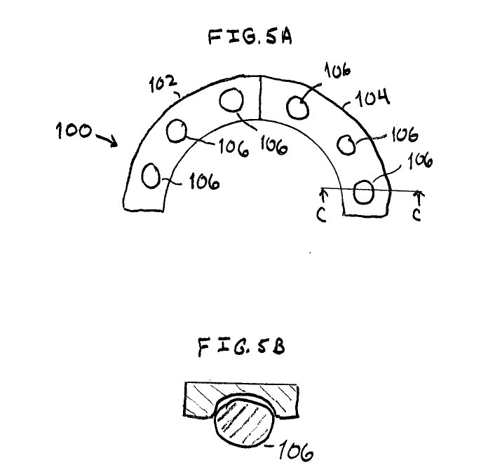

FIG. 5A and FIG. 5B illustrate a second embodiment of the present invention in which an upper part (100), preferably comprising two halves (102, 104), has a series of raised, semispherical bumps (106) instead of the raised ridge described in the previous embodiment. It is otherwise identical to the first embodiment in all other respects. The bumps (106) are in an array that forms an arcuate path to cooperate with a lower part with groove that is identical to that described with respect to the first embodiment.

When the upper and lower parts (22, 24) of the first embodiment, or the second embodiment, are inserted into a patient, the patient's natural bending and twisting movement are accommodated by the relative movement of the upper and lower parts as described herein. Although not limited thereto, the present invention is particularly well-suited for posterior approach procedures when each of the upper and lower parts (22, 24) are provided in two or more parts that can be inserted individually and assembled in place.

While the preferred embodiments have been herein shown and described, it is understood that various modification can be made without departing from the scope of the present invention.

What is claimed is:

1) An implantable vertebral motion preservation system comprising
a first part having a first end face adapted to engage a first vertebral body;
a second part having a first end face adapted to engage a second vertebral body, said second vertebral body being adjacent to said first vertebral body;
a generally arcuate ridge portion on said first part located on a surface of said first part that is opposite of said first vertebral body and that is generally horizontal, said ridge extending in a direction toward said second vertebral body; and
a generally arcuate groove portion on said second part located on a surface of said second part that is opposite of said second vertebral body and that is generally horizontal, said groove being sized to receive said ridge.

2) A system according to claim 1, wherein
said ridge and said groove are adapted to slide relative to each other, along said respective arcuate paths, to facilitate relative rotation between said first part and said second part in a generally horizontal plane.

3) A system according to claim 1, wherein
said ridge has a generally semi-spherically shaped cross-sectional profile, and said groove has a generally concave cross-sectional profile.

4) A system according to claim 1, wherein
at least one of said first part and said second part comprises at least two parts that can selectively be assembled and disassembled.

5) A system according to claim 3, wherein
said ridge and said groove are adapted to slide relative to each other along an arcuate path defined by said concave profile of said groove to facilitate relative rotation between said first part and said second part in a generally vertical plane.

6) An implantable vertebral motion preservation system comprising
a first part having a first end face adapted to engage a first vertebral body;
a second part having a first end face adapted to engage a second vertebral body, said second vertebral body being adjacent to said first vertebral body;
a generally arcuate array of raised semi-spherical bumps on said first part located on a surface of said first part that is opposite of said first vertebral body and that is generally horizontal, said array extending in a direction toward said second vertebral body; and
a generally arcuate groove portion on said second part located on a surface of said second part that is opposite of said second vertebral body and that is generally horizontal, said groove being sized to receive said array.

7) A system according to claim 6, wherein
said array and said groove are adapted to slide relative to each other, along said respective arcuate paths, to facilitate relative rotation between said first part and said second part in a generally horizontal plane.

8) A system according to claim 6, wherein
each said semi-spherical bump has a generally semi-spherically shaped cross-sectional profile, and said groove has a generally concave cross-sectional profile.

9) A system according to claim 6, wherein
at least one of said first part and said second part comprises at least two parts that can selectively be assembled and disassembled.

10) A system according to claim 8, wherein
each said bump and said groove are adapted to slide relative to each other along an arcuate path defined by said concave profile of said groove to facilitate relative rotation between said first part and said second part in a generally vertical plane.
